(19) United States
(12) Patent Application Publication
(10) Pub. No.: US 2005/0049613 A1
(43) Pub. Date: Mar. 3, 2005

(54) In vivo deflection device and method

(76) Inventor: Joe D. Brown, Panama City Beach, FL (US)

Correspondence Address: BACON & THOMAS, PLLC, 625 SLATERS LANE, FOURTH FLOOR, ALEXANDRIA, VA, 22314, US

(21) Appl. No.: 10/875,223

(22) Filed: Jun. 25, 2004

Related U.S. Application Data
(60) Provisional application No. 60/482,454, filed on Jun. 26, 2003.

(51) Int. Cl. A61B 17/22

(52) U.S. Cl. 606/127

(57) ABSTRACT

A surgical instrument such as a basket or grasper is advanced toward an obstruction until it extends past the obstruction, at which time memory wires in the grasper are heated by warm water, gas, electricity, or the like, to cause the wires to deflect around and grasp the obstruction. If it is desired to release the obstruction, water having a lower temperature may be applied to cool the wires.

This application claims the benefit of U.S. Provisional Application Ser. No. 60/482,454, filed Jun. 26, 2003.

### BACKGROUND OF THE INVENTION

1. Field of the Invention

This invention relates to an in vivo deflection device and method, and more particularly to:
1. use of shape memory alloy structures to open and close a stone basket or grasper; and
2. more generally, a method and apparatus for actively causing deflection of a shape memory alloy structure to manipulate an instrument such as a basket, fiber, or cone from outside an endoscope or catheter that has been inserted into a patient by changing the temperature of a shape memory alloy using electrical wires, fluid-carrying tubes, or other means of causing a change in temperature that are accessible from outside the patient's body.

Changing the temperature of the shape memory alloy to open and close the bucket or grasper, or to otherwise manipulate a surgical instrument from outside an endoscope or grasper, may be carried out, for example, by causing a fluid to flow through the alloy structure, by applying electricity directly to the structure or to a heating element within the structure, or any other means of causing internal heating of the shape memory allow structure that can be triggered or controlled from outside the patient's body.

2. Description of Related Art

U.S. patent application Ser. No. 10/153,895, filed May 24, 2002, and incorporated by reference herein, discloses use of a shape memory alloy to deflect a surgical instrument inserted into an endoscope, deflection of the shape memory alloy being controlled by temperature of an irrigation fluid.

U.S. Provisional Patent Application Ser. No. 60/416,588, filed Oct. 8, 2002, and also incorporated by reference herein, extends the concept of using the temperature of an irrigation fluid to deflect a shape memory alloy to surgical instruments that need not be inserted into endoscopes, such as a urological retrieval coil used to trap or retrieve stones formed in the urological tract.

U.S. Provisional Application Ser. No. 60/451,283 further expands the concept using shape memory alloy structures for in vivo instrument manipulation to include activation means other than temperature-controlled fluids, including electrical activation, and discloses application of the concept to clamps used to stop leakage of blood vessels, and to repair other body parts, after surgery has been completed.

### SUMMARY OF THE INVENTION

The present application extends the concept of in vivo instrument deflection to cover stone baskets or graspers, which are used to retrieve obstructions in the urinary tract, kidneys, or other organs or systems, and to other instruments extending outside an endoscope or catheter, by using temperature-controlled fluids, electricity, other means of deflecting shape memory alloy structures in the instruments.

A key feature of the invention is that the shape memory structures are actively deflected from outside the patient, either electrically, by means of fluids passed through or around the device, or other means such as radiation that can cause heating or, more generally, a change in temperature of the device, including any of the heating means disclosed in the above-cited U.S. Provisional Patent Application Ser. No. 60/451,283.

When the invention is applied to a stone basket or grasper, for example, the basket or grasper is advanced toward the obstruction until tines on the basket or grasper extend past the obstruction, at which time memory wires in the grasper may be heated by warm water through the channel of an endoscope or catheter, by electricity, or by other means, to cause the wires to deflect around and grasp the obstruction. If it is desired to release the obstruction, the wires are simply cooled to release the grasper, by application of cool water or other means, at which time the grasper may be pulled back into the endoscope or catheter.

Finally, the shape memory structure of the invention may be in the form of Nitinol wires similar to those disclosed in the above-cited U.S. patent application Ser. No. 10/153,895.

### BRIEF DESCRIPTION OF THE DRAWINGS

FIG. 1 is a schematic drawing of a stone grasping arrangement constructed in accordance with the principles of a preferred embodiment of the invention.

FIG. 2 is a schematic diagram of the grasping arrangement of FIG. 1, before grasping of the stone.

FIG. 3 is a schematic diagram of the grasping arrangement of FIG. 1, after grasping of the stone.

FIG. 4 is a schematic diagram of the grasping arrangement of FIG. 1, upon release of the stone

### DETAILED DESCRIPTION OF THE PREFERRED EMBODIMENTS

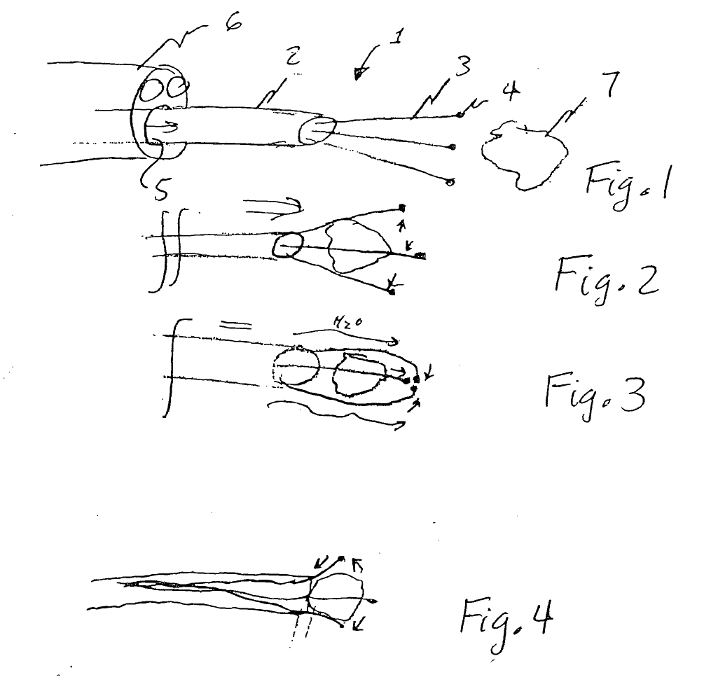

As shown in FIG. 1, an in vivo deflection device in the form of a stone basket or grasper 1 includes a shaft 2 and deflectable tines 3 terminating in bull tips 4.

In use, grasper 1 and tines 3 are inserted through a channel 5 of an endoscope or catheter 6, or inserted directly into the body of the patient in parallel with the endoscope or catheter, and advanced beyond the end of the endoscope or catheter to a position, shown in FIG. 2, at which the tines 3 may grasp a stone or other obstruction 7. The tines 3 are then caused to deflect around the stone or obstruction 7, as shown in FIG. 3, by heating the tines. The tines may be heated by causing water having a temperature greater than body temperature to flow from channel 5 around the tines, by electrically heating the tines, by application of a gas, or by any other means of causing a shape memory alloy to assume a predetermined shape.

In order to release the stone or other obstruction 7, it is obviously necessary to stop application of heat, whether by water or other means. To expedite release, relatively cool water may be applied through channel 5 of the endoscope or catheter 6. The application of cool water to enable immediate release may be used even where heating is by electrical or other means. In theory, it may also be possible to cool the tines by other means, including application of a gas, although use of cool water appears to be simplest.

The in vivo deflection device of the preferred embodiment is, of course, not limited to a stone basket or grasper and, as discussed in the above cited prior provisional and regular U.S. patent applications, and may be made of or include any shape memory alloy, including but not limited to Nitinol, that deflects or twists at a transformation temperature that is above body temperature, but that is not so warm that it would cause damage to the patient when the device is heated. As is well-known, the coil or other shape is determined during manufacture of the instrument by forming the alloy into the desired shape at high temperature, and subsequently cooling and deforming the alloy as it cools.

Having thus described a preferred embodiment of the invention in sufficient detail to enable those skilled in the art to make and use the invention, it will nevertheless be appreciated that numerous variations and modifications of the illustrated embodiment may be made without departing from the spirit of the invention, and it is intended that the invention not be limited by the above description or accompanying drawings, but that it be defined solely in accordance with the appended claims.

1. A stone basket or grasper for insertion into a patient, said stone basket or grasper comprising a plurality of tines arranged to extend around and grasp an obstruction, wherein the plurality of tines include shape memory alloy structures, and wherein the shape memory structures are arranged to be actively deflected from outside a patient by heating the shape memory structures and causing the shape memory alloy structures to assume a predetermined shape.

2. The stone basket or grasper of claim 1, wherein the shape memory structures are arranged to be electrically heated in order to cause the shape memory alloy structures to assume the predetermined shape.

3. The stone basket or grasper of claim 1, wherein the shape memory structures are arranged to be heated by causing water having a temperature greater than body temperature to flow through a channel around the tines in order to cause the shape memory alloy structures to assume the predetermined shape.

4. The stone basket or grasper of claim 1, wherein the shape memory structures are arranged to be heated by in order to cause the shape memory alloy structures to assume the predetermined shape.

5. The stone basket or grasper of claim 1, wherein the tines are arranged to release the obstruction upon cessation of said application of heat.

6. The stone basket or grasper of claim 5, wherein said release is expedited by application of cool water.

7. The stone basket or grasper of claim 1, wherein the shape memory structures are made of Nitinol having a transformation temperature that is above body temperature.

8. A method of grasping an obstruction within a patient, comprising the steps of:
advancing a basket or grasper is advanced toward the obstruction until tines on the basket or grasper extend past the obstruction; and
actively heating shape memory alloy structures in the tines until the tines assume a predetermined shape and are deflected to extend around the obstruction.

9. A method as claimed in claim 8, wherein the shape memory alloy structures are electrically heated to deflect the tines around the obstruction.

10. A method as claimed in claim 8, wherein the shape memory alloy structures are heated by causing water having a temperature greater than body temperature to flow from a channel around the tines.

11. A method as claimed in claim 8, further comprising the step of releasing the obstruction by stopping application of heat.

12. A method as claimed in claim 11, further comprising the step of expediting release by causing cool water to flow from a channel around the tines.
